(19) United States
(12) Patent Application Publication
(10) Pub. No.: US 2014/0296789 A1
(43) Pub. Date: Oct. 2, 2014

(54) Multi-Lumen Needle and Catheter Guidance System

(71) Applicants: Kody El-Mohtar, Albany, NY (US); Eric Ledet, Guilderland, NY (US)

(72) Inventors: Kody El-Mohtar, Albany, NY (US); Eric Ledet, Guilderland, NY (US)

(21) Appl. No.: 13/851,241

(22) Filed: Mar. 27, 2013

(51) Int. Cl. A61M 25/00

(52) U.S. Cl. CPC A61M 25/0084
USPC 604/173

(57) ABSTRACT

A multi-lumen needle and catheter guidance system is provided and includes: a multi-lumen needle including a pair of rectangular lumens, the pair of rectangular lumens extending longitudinally within and through a shaft of the multi-lumen needle from a proximal end of the multi-lumen needle to a distal end of the multi-lumen needle, the pair of rectangular lumens diverging in opposite directions and ending at a pair of rectangular apertures formed on opposing sides of the distal end of the multi-lumen needle; and a pair of rectangular catheters for insertion into and through the pair of lumens, wherein distal ends of the rectangular catheters are configured to pass out of the distal end of the multi-lumen needle in opposing directions through the pair of rectangular apertures.

### TECHNICAL FIELD

The present invention is directed to medical devices. More particularly, the present invention is directed to a multi-lumen needle and catheter guidance system.

### RELATED ART

Pregnancy labor is characterized by regular, painful uterine contractions that increase in frequency and intensity and are associated with progressive cervical effacement and dilatation.

Labor has been divided into three stages. The first stage occurs from onset of cervical change to 10 cm dilatation. It can be divided into latent and accelerative phases. The latent phase can last up to 8 hours, while the accelerative phase is associated with a faster rate of cervical dilatation and usually begins at 2-4 cm dilatation and the duration varies from 2 to 6 hours.

The second stage occurs from full cervical dilatation (10 cm) to delivery of the baby. The third stage begins right after the birth of the baby and ends with the delivery of the placenta.

Pain during the first stage of labor is due to uterine contractions and stretching of the cervix. It is cramping and visceral in nature, diffuse and poorly localized. Sensations are carried through primary afferent fibers which pass sequentially through the inferior, middle and superior hypogastric plexus, the lumbar and lower thoracic sympathetic chain and end in rami communicantes associated with T10-L1 spinal nerves.

During the late first and second stage of labor, somatic pain predominates, as a result of distension and traction on the pelvic structures, the pelvic floor and the perineum and is carried via the pudendal nerve. Unlike the visceral pain of the first stage, the pain is sharp and well localized, due mainly to less arborization and the faster conduction velocity in the sacral pathways.

The experience of labor pain is different for each woman, and the different methods chosen to relieve pain depend upon the techniques available locally and the personal choice of the individual. Two common pain relief methods include a lumbar epidural block and a caudal epidural block in which anesthetic compounds are introduced into different areas of the epidural space.

### SUMMARY

Embodiments of the invention are directed to a multi-lumen needle and catheter guidance system. The invention can be used, for example, for administration of compounds (e.g., anesthetic compounds) into the epidural space of a patient. In an embodiment, one catheter may be guided in a first direction (e.g., cranially) by the multi-lumen needle to provide a lumbar block, while another catheter may be guided by the multi-lumen needle in a second, opposite direction (e.g., caudally) to provide a caudal block. This allows one needle stick in the patient's back to achieve both blocks.

One aspect of the invention is directed to a multi-lumen needle and catheter guidance system, comprising: a multi-lumen needle including a pair of rectangular lumens, the pair of rectangular lumens extending longitudinally within and through a shaft of the multi-lumen needle from a proximal end of the multi-lumen needle to a distal end of the multi-lumen needle, the pair of rectangular lumens diverging in opposite directions and ending at a pair of rectangular apertures formed on opposing sides of the distal end of the multi-lumen needle; and a pair of rectangular catheters for insertion into and through the pair of lumens, wherein distal ends of the rectangular catheters are configured to pass out of the distal end of the multi-lumen needle in opposing directions through the pair of rectangular apertures.

### BRIEF DESCRIPTION OF THE DRAWINGS

Embodiments of the present invention will now be described by way of example with reference to the accompanying drawings in which like references denote similar elements.

FIG. 1 depicts a multi-lumen needle and catheter guidance system according to embodiments.

FIG. 2 depicts a proximal end of the multi-lumen needle of FIG. 1 according to embodiments.

FIG. 3 depicts a distal end of the multi-lumen needle of FIG. 1 according to embodiments.

FIG. 4 depicts the insertion of catheters into the multi-lumen needle of FIG. 1 according to embodiments.

FIGS. 5 and 6 depict the exiting of catheters from the multi-lumen needle of FIG. 1.

FIG. 7 depicts an operational view of the multi-lumen needle system and catheter guidance system of FIG. 1.

### DETAILED DESCRIPTION

The invention is a multi-lumen needle and catheter guidance system. In embodiments, the invention can be used, for example, for administration of compounds (e.g., anesthetic compounds) into the epidural space of a patient. However, one skilled in the art would recognize that the multi-lumen needle and catheter guidance system of the invention may also be used to provide venous access, arterial access, or access to other cavities or spaces inside a body, for the administration of compounds or for other purposes.

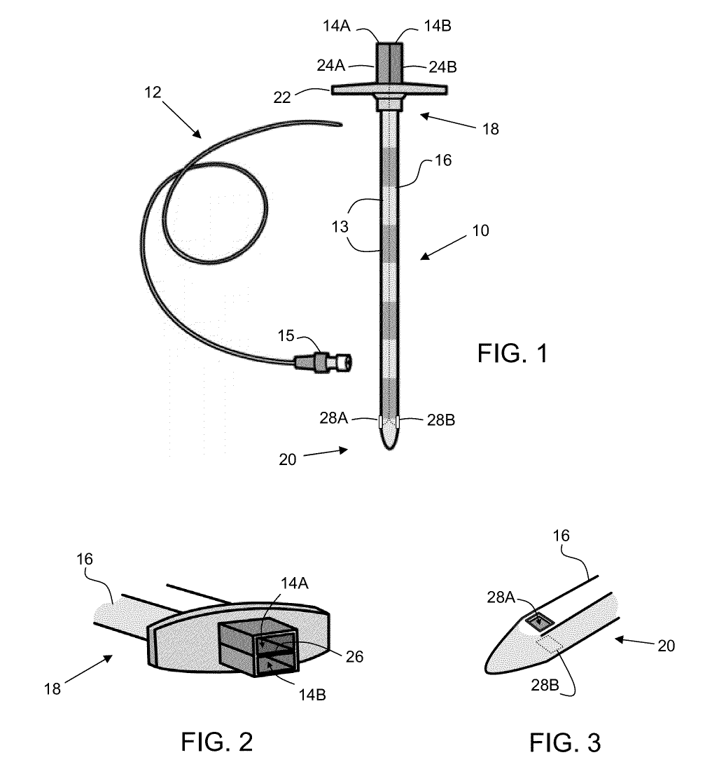

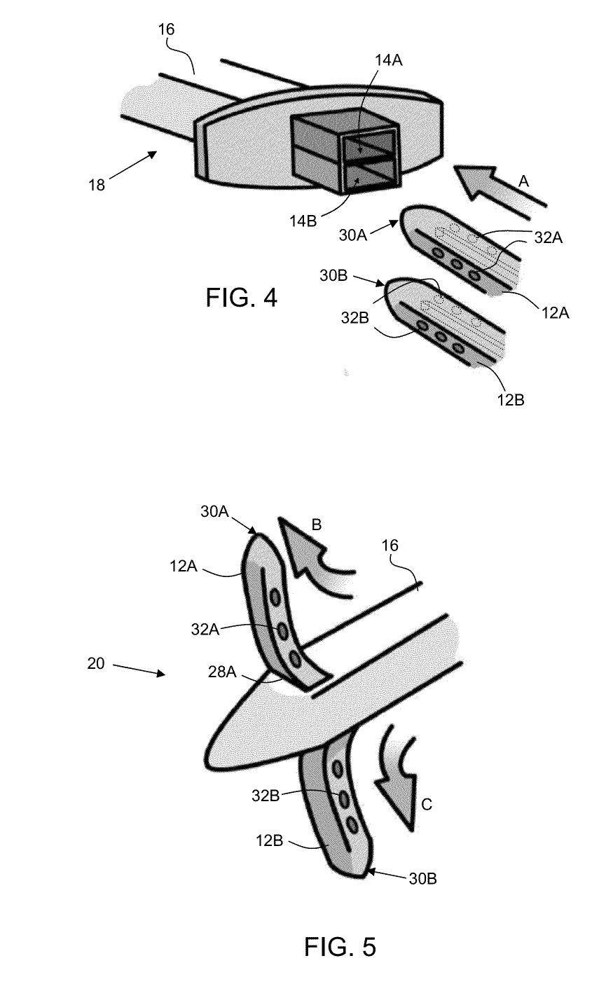

As depicted in FIG. 1, embodiments of the multi-lumen needle and catheter guidance system of the present invention include a multi-lumen needle 10 and at least one catheter 12. The multi-lumen needle 10 may be provided with a plurality of lumens 14A, 14B. Although more than two lumens 14A, 14B may be provided in various embodiments, the description below will be directed to a multi-lumen needle 10 having a first lumen 14A and a second lumen 14B. However, the functionality of the multi-lumen needle and catheter guidance system of the present invention can easily be extended to accommodate more than two lumens and associated catheters. The lumens 14A, 14B serve to guide respective catheters 12A, 12B (FIGS. 4-6) for insertion into and through the multi-lumen needle 10 and into the body of a patient.

The lumen 14A extends longitudinally through a shaft 16 of the multi-lumen needle 10 from approximately a proximal end 18 of the multi-lumen needle 10 to approximately a distal end 20 of the multi-lumen needle 10. Similarly, the lumen 14B extends longitudinally through the shaft 16 of the multi-lumen needle 10 from approximately the proximal end 18 of the multi-lumen needle 10 to approximately the distal end 20 of the multi-lumen needle 10. Flanges 22 are provided near the proximal end 18 of the multi-lumen needle 10 to allow the multi-lumen needle 10 to be grasped, positioned, and manipulated. A portion 24A of the lumen 14A and a portion 24B of the lumen 14B may extend outside of the shaft 16 of the multi-lumen needle 10 at the proximal end 18 of the multi-lumen needle 10. The portions 24A, 24B of each lumen 14A, 14B that extend outside of the shaft 16 of the multi-lumen needle 10 may be color coded and/or may include other identifying indicia to assist in differentiating between the lumens 14A, 14B.

As illustrated in greater detail in FIG. 2, the lumens 14A, 14B may be separated from each other within the shaft 16 of the multi-lumen needle 10. Each lumen 14A, 14B may extend longitudinally through the shaft 16 of the multi-lumen needle 10 from approximately the proximal end 18 of the multi-lumen needle 10 to approximately the distal end 20 of the multi-lumen needle 10. In embodiments, separate lumens 14A, 14B may be provided and positioned such that adjacent side walls of the lumens 14A, 14B separate the lumens 14A, 14B at least along the length of the shaft 16 of the multi-lumen needle 10. A single lumen with a center divider 26 may also be used to form and separate the lumens 14A, 14B. The lumens 14A, 14B may be configured to have the same opening shape and size as depicted in FIG. 2, or may be configured to have different opening shapes and/or sizes.

In embodiments, as depicted in FIG. 1, the lumens 14A, 14B run parallel to each other and parallel to the longitudinal axis of the shaft 16 of the multi-lumen needle 10. As the lumens 14A, 14B approach the distal end 20 of the multi-lumen needle 10, the lumens 14A, 14B diverge from each other in different (e.g., opposing) direction and terminates at a corresponding aperture 28A, 28B formed through the sidewalls of the shaft 16 of the multi-lumen needle 10. To this extent, the lumens 14A, 14B exit the shaft 16 of the multi-lumen needle 10 oriented in different (e.g., opposite) directions. An enlarged view of the apertures 28A, 28B is shown in FIG. 3. In other embodiment, more than two apertures may be present in the multi-lumen needle 10.

The distal end 20 of the multi-lumen needle 10 may be shaped such that it substantially comes to a point to foster penetration through the skin, fascia, dura, or other soft tissue of a patient. In embodiments, the shape of the distal end 20 of the multi-lumen needle 10 may be conical, “bullet” shaped, beveled, and/or the like. A non-limiting example of a shape of the distal end 20 of the multi-lumen needle 10 is depicted in FIG. 3.

In embodiments, the multi-lumen needle 10 may be fabricated from a biocompatible metal, but it may also be fabricated from a polymer, ceramic, or composite material. The multi-lumen needle 10 may also include features that foster handling and manipulation during insertion. This may include a handle on the proximal end 18 of the multi-lumen needle 10, or other features to enhance manipulation of the multi-lumen needle 10 by the a user, such as traction grooves. The multi-lumen needle 10 may also have depth indicators 13 to foster identification of its depth of penetration. The multi-lumen needle 10 may also have connections 15 on the distal end of each lumen 14A, 14B to interface with tubing or syringes, such as a Luer lock connection, tapered connection, or other connection to facilitate connection with syringes or tubing. The multi-lumen needle 10 may be of any suitable length or size. For instance, the multi-lumen needle 10 may have a size of 14-17 French, but could also be larger or smaller for different applications.

Each lumen 14A, 14B is specifically configured to facilitate the deployment of a corresponding catheter 12A, 12B (FIGS. 4-7) toward and out of a corresponding aperture 28A, 28B in the distal end 20 of the multi-lumen needle 10. For instance, as shown for example in FIG. 2, each lumen 14A, 14B may have a rectangular cross-section, with a major axis oriented transversely and a minor axis oriented axially. In other embodiments, the lumens 14A, 14B may also be square, ovoid, elliptical, round, or any other shape that fosters catheter deployment. In the illustrated embodiment, the aperture 28A is located at an angle of approximately 180 degrees from the aperture 28B. One skilled in the art should recognize the apertures 28A, 28B at the distal end 20 of the multi-lumen needle 10 may be oriented at any relative angle.

In embodiments, as depicted in FIGS. 4-7, the configuration of the lumens 14A, 14B, together with the orientation and shape of the apertures 28A, 28B and the configuration of the catheters 12A, 12B, helps to direct each catheter 12A, 12B in a specific direction (e.g., cranially and caudally) away from the distal end 20 of the multi-lumen needle 10. In embodiments, each catheter 12A, 12B may have a cross-sectional shape that matches the cross-sectional shape of a corresponding lumen 14A, 14B. For example, if a lumen 14A, 14B has a rectangular cross-section, then each catheter 12A, 12B may also have a rectangular cross-section (but would be of a smaller size such that the catheters 12A, 12B can be longitudinally displaced within a corresponding lumen 14A, 14B). This, for example, helps a catheter 12A, 12B follow the path of a corresponding lumen 14A, 14B in, through, and out of the multi-lumen needle 10. The cross-sectional geometry of each catheter 12A, 12B may be uniform along the length of the catheter 12A, 12B or may be variable along its length, as long as the catheter 12A, 12B can pass within the cross-sectional geometry of the lumens 14A, 14B in the multi-lumen needle 10.

In embodiments, each aperture 28A, 28B may have a cross-sectional shape that matches the cross-sectional shape of a corresponding lumen 14A, 14B and/or catheter 12A, 12B. For example, if a lumen 14A, 14B and/or a catheter 12A, 12B has a rectangular cross-section, then each aperture 28A, 28B may also have a rectangular cross-section. The rectangular cross-section of the apertures 28A, 28B may be the same size as or larger than the rectangular cross-section of the lumens 14A, 14B.

Referring now specifically to FIG. 4, the insertion of the catheters 12A, 12B into the multi-lumen needle 10 is shown. In particular, an end 30A of the catheter 12A and an end 30B of the catheter 12B may be inserted into a respective lumen 14A, 14B as indicated by arrow A. The end 30A, 30B of each catheter 12A, 12B has a geometry and a plurality of openings 32A, 32B that will be discussed in greater detail below.

FIG. 5 illustrates the multi-lumen needle 10 after the catheters 12A, 12B have been inserted through the lumens 14A, 14B. As shown, the end 30A, 30B of each catheter 12A, 12B has passed outward through a corresponding aperture 28A, 28B in the distal end 20 of the multi-lumen needle 10. Each catheter 12A, 12B can be manipulated independently to allow the end 30A, 30B of each catheter 12A, 12B to be selectively and independently positioned in a patient at a desired distance (or different distances) from the distal end 20 of the multi-lumen needle 10. This allows, for example, the same or different compounds (e.g., anesthetic compounds) to be independently dispensed at different locations within a patient via a single multi-lumen needle 10. Once the catheters 12A, 12B are suitably deployed within the patient, the multi-lumen needle 10 can be removed over the catheters 12A, 12B, thereby leaving the catheters 12A, 12B in place within the patient.

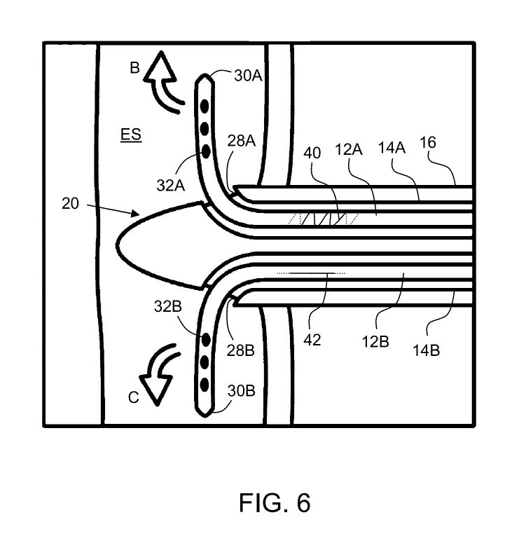

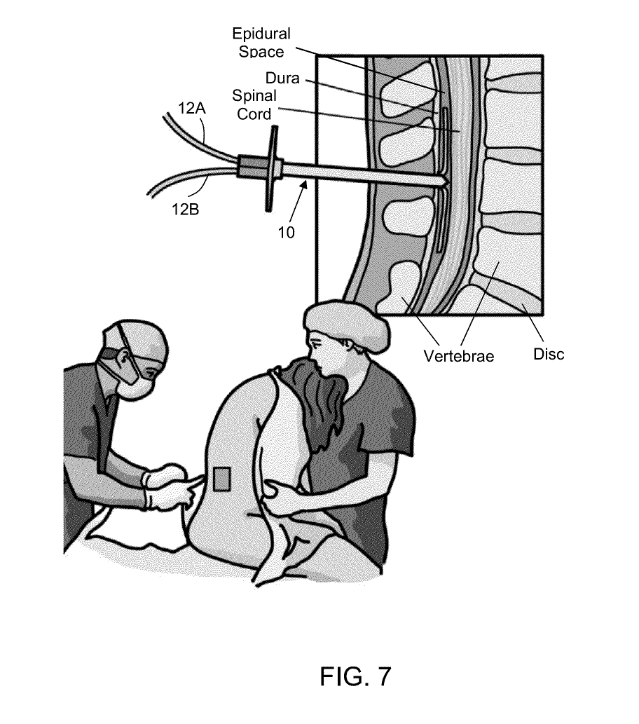

In embodiments, as shown in FIGS. 6 and 7, after insertion of the multi-lumen needle 10 into the epidural space (ES) of a patient, the shaft 16 of the multi-lumen needle 10 may be oriented such that one aperture (e.g., aperture 28A) is direct caudally, while another aperture (e.g., aperture 28B) is directed cranially. In such a case, the catheter 12A may be inserted into the lumen 14A at the proximal end 18 of the multi-lumen needle 10 such that it passes through the shaft 16 and exits the distal end 20 of the multi-lumen needle 10 via the aperture 28A. The end 30A of the catheter 12A is thus directed caudally through the aperture 28A as indicated by arrow B. Similarly, the catheter 12B may be inserted into the lumen 14B at the proximal end 18 of the multi-lumen needle 10 such that it passes through the shaft 16 and exits the distal end 20 of the multi-lumen needle 10 via the aperture 28B. The end 30B of the catheter 12B is thus directed cranially through the aperture 28B as indicated by arrow C.

The catheters 12A, 12B may have a cross-sectional geometry that facilitates their passage into and through the lumens 14A, 14A of the multi-lumen needle 10 and out of the apertures 28A, 28B formed in the distal end 20 of the multi-lumen needle 10, without binding, kinking, or otherwise failing during passage. In embodiments, for example as depicted in FIGS. 4 and 5, the catheters 12A, 12B have a rectangular cross-section with a major axis oriented transversely and a minor axis oriented axially. In general, the cross-sectional geometry of the catheters 12A, 12B matches the cross-sectional geometry of the lumens 14A, 14B. This helps to foster deployment of the catheters 12A, 12B in opposite (e.g., cranial and caudal) directions as the catheters 12A, 12B pass through the apertures 28A, 28B formed in the distal end 20 of the multi-lumen needle 10. In other embodiments, the cross-sectional geometry of the catheters 12A, 12B may be square, ovoid, elliptical, round, or any other geometry that fosters catheter deployment.

In embodiments, for example as depicted in FIG. 4, each catheter 12A, 12B includes a respective lumen 34A, 34B, which runs the length of the catheter 12A, 12B and is oriented along the axial direction of the catheter 12A, 12B. The lumen 34A is in fluid communication with the corresponding plurality of openings 32A formed near the end 30A of the catheter 12A. Similarly, the lumen 34B is in fluid communication with the corresponding plurality of openings 32B formed near the end 30B of the catheter 12B. The openings 32A, 32B are provided on at least one side of the catheters 12A, 12B and may be oriented perpendicular to the direction of insertion of the catheters 12A, 12B into the epidural space of the patient. The openings 32A, 32B may have any suitable cross-sectional shape (e.g., circular, oval, etc.), and may be oriented in any suitable pattern to facilitate a uniform or non-uniform distribution and release of fluids into the epidural space.

As depicted in FIGS. 4-6, the end 30A, 30B of each catheter 12A, 12B may be shaped such that it substantially comes to a point to foster passage through the skin, fascia, dura, or other soft tissue. The end 30A, 30B of each catheter 12A, 12B may, for example, be conical, “bullet” shaped, or have a beveled shape. In embodiments in which the catheters 12A, 12B do not need to penetrate through tissue, the ends 30A, 30B may be blunted.

The catheters 12A, 12B may be fabricated from a biocompatible material such as a polymer or a metal. Ceramic or composite materials may also be used. The catheters 12A, 12B may be formed from a radiolucent material to allow visualization during insertion (e.g., using x-ray imagery).

The interior, exterior, and/or walls of the catheters 12A, 12B can be reinforced or otherwise configured to facilitate the deployment and operation of the catheters 12A, 12B. For example, as depicted in FIG. 6, an element (e.g., a wound metal sleeve 40) may be provided along at least a portion of the length of the catheters 12A, 12B (e.g., extending up to about 6 cm from the ends 30A, 30B of the catheters 12A, 12B). In another embodiment, an element (e.g., a wire 42) may formed of a shape-memory alloy (e.g., copper-aluminium-nickel, nickel-titanium (NiTi), and/or the like) that “remembers” its original, cold-forged shape. The wire 42 may be provided along (e.g., embedded within) at least a portion of the length of the catheters 12A, 12B (e.g., extending up to about 6 cm from the ends 30A, 30B of the catheters 12A, 12B). In this case, the wire 42 can cause the catheters 12A, 12B to have a 90 degree curve prior to insertion into the multi-lumen needle 10. The wire 42 is temporarily straightened as it passes though the multi-lumen needle 10, returning to its original shape as it passes out of the multi-lumen needle 10 into a patient. In either case, the catheters 12A, 12B is compliant enough to bend as it follows the path of the lumens 14A, 14B in the multi-lumen needle 10, but rigid enough so that it will not kink, buckle, or otherwise fail during passage through the multi-lumen needle 10 and into a patient.

The foregoing description of various aspects of the invention has been presented for purposes of illustration and description. It is not intended to be exhaustive or to limit the invention to the precise form disclosed, and obviously, many modifications and variations are possible. Such modifications and variations that may be apparent to an individual in the art are included within the scope of the invention as defined by the accompanying claims.

1. A multi-lumen needle and catheter guidance system, comprising:
a multi-lumen needle including a pair of rectangular lumens, the pair of rectangular lumens extending longitudinally within and through a shaft of the multi-lumen needle from a proximal end of the multi-lumen needle to a distal end of the multi-lumen needle, the pair of rectangular lumens diverging in opposite directions and ending at a pair of rectangular apertures formed on opposing sides of the distal end of the multi-lumen needle; and
a pair of rectangular catheters for insertion into and through the pair of lumens, wherein distal ends of the rectangular catheters are configured to pass out of the distal end of the multi-lumen needle in opposing directions through the pair of rectangular apertures.

2. The multi-lumen needle and catheter guidance system of claim 1, wherein fluids are administered into a body cavity of a patient through the pair of rectangular catheters.

3. The multi-lumen needle and catheter guidance system of claim 2, wherein the body cavity comprises an epidural space of a patient, and wherein a first one of the pair of rectangular catheters is directed cranially and a second one of the pair of rectangular catheters is directed caudally through the pair of rectangular apertures.

4. The multi-lumen needle and catheter guidance system of claim 1, wherein the rectangular lumens, rectangular catheters, and rectangular apertures foster deployment of the rectangular catheters within a patient without failure or binding of the rectangular catheters.

5. The multi-lumen needle and catheter guidance system of claim 2, wherein the rectangular catheters include at least one opening through which fluids can be administered into a body cavity of a patient.

6. The multi-lumen needle and catheter guidance system of claim 5, wherein the at least one opening is oriented perpendicular to a direction of insertion of the pair of rectangular catheters into the body cavity of a patient.

7. The multi-lumen needle and catheter guidance system of claim 1, wherein a distal end of the rectangular catheters has a pointed configuration.

8. The multi-lumen needle and catheter guidance system of claim 1, wherein the pair of rectangular catheters can be deployed independently and at different distances within a body cavity of a patient.

9. The multi-lumen needle and catheter guidance system of claim 1, wherein the pair of rectangular catheters include a reinforcing element.

10. The multi-lumen needle and catheter guidance system of claim 1, wherein the pair of rectangular catheters include a shape-memory element.
